(19) United States
(12) Patent Application Publication
(10) Pub. No.: US 2026/0263692 A1
(43) Pub. Date: Sep. 10, 2026

(54) Drug delivery control arrangement, and injector

(71) Applicant: SHL Medical AG, Zug (CH)

(72) Inventors: Daniel Scott, Deerfield Beach, FL (US); Torbjörn Neby, Nacka Strand (SE); Hsuan Wang, Zug (CH); Plamen Balkandjiev; Christoph Egloff, Zug (CH)

(21) Appl. No.: 18/871,886

(22) PCT Filed: May 26, 2023
(86) PCT No.: PCT/EP2023/064171

§ 371 Date: Dec. 5, 2024

Related U.S. Application Data
(60) Provisional application No. 63/350,478, filed on Jun. 9, 2022.

(30) Foreign Application Priority Data
Sep. 14, 2022 (EP) 22195733.5

(51) Int. Cl. A61M 5/152; A61M 5/142; A61M 5/172

(52) U.S. Cl. CPC A61M 5/152; A61M 5/14244; A61M 5/172; A61M 2205/33

(57) ABSTRACT

The present disclosure provides a drug delivery control arrangement configured to enable medicament delivery from at least two medicament bags. Each one of the medicament bags has a corresponding gear associated for facilitating the medicament to be delivered from that medicament bag. The drug delivery control arrangement comprises a drive motor configured to be engaged with the gears, one at a time, for delivering the medicament out of the corresponding medicament bags. The drug delivery control arrangement further comprises a control unit configured to control from which one of the medicament bags the medicament is delivered by engaging the drive motor with the gear corresponding to that medicament bag.

### CROSS-REFERENCE TO RELATED APPLICATIONS

The present application is a U.S. National Phase Application pursuant to 35 U.S.C. § 371 of International Application No. PCT/EP 2023/064171 filed May 26, 2023, which claims priority to (i) U.S. Provisional Application No. 63/350,478 filed Jun. 9, 2022, and (ii) EP Patent Application No. 22195733.5 filed Sep. 14, 2022. The entire disclosure contents of these applications are herewith incorporated by reference into the present application.

### TECHNICAL FIELD

This disclosure relates to medical devices, especially to arrangements for handling medicament bags for injectors.

### BACKGROUND

Medicaments may be administered to patients in various ways, e.g. through the mouth, either via the digestive channel or by inhalation or by injection. The focus of this disclosure is injections. The medicaments to be administered to the patient are provided in medicament bags from which they are pumped into a patient. The administration may be conducted by medical staff, like nurses or doctors in hospital, but also by patients themselves, e.g. at home.

It is a challenge to achieve an effective and convenient delivery of medicaments, and the applicant has appreciated that it would be desirable to improve performance of medical injectors.

### SUMMARY

According to a first aspect, a drug delivery control arrangement configured to enable medicament delivery from at least two medicament bags is provided. Each one of the medicament bags has a corresponding gear associated for facilitating the medicament to be delivered from that medicament bag. The drug delivery control arrangement comprises: a drive motor configured to be engaged with the gears, one at a time, for delivering the medicament out of the corresponding medicament bags; and a control unit configured to control from which one of the medicament bags the medicament is delivered by engaging the drive motor with the gear corresponding to that medicament bag. This can provide a mechanism that facilitates administration of medicaments from a plurality of medicament bags.

The control unit may comprise a transfer mechanism configured to move the drive motor between at least two positions, wherein for each of the positions, the drive motor is engaged with different ones of the gears. The transfer mechanism may comprise at least one of: a control motor, a spring, and a solenoid. The transfer mechanism comprises at least one of: a belt; a wire; a toothed rack; and a combination of stops and flexible arms.

The drug delivery control arrangement may comprise a sensor configured to measure a parameter related to the currently delivering medicament bag when in use. The control unit may be configured to initiate movement of the drive motor between the at least two positions based on the measured parameter. Preferably, the measured parameter is one of: a flow rate out of the currently delivering medicament bag; a torque of the drive motor, and a dimension of the currently delivering medicament bag. The control unit may be configured to determine a status of the currently delivering one of the medicament bags based on the measured parameter and initiate the movement of the drive motor based on the determined status. The status may be one of: remaining drug volume in the currently delivering medicament bag; remaining drug quantity in the currently delivering medicament bag; and remaining injection time.

By the implemented mechanism, a convenient arrangement for delivery of medicaments from multiple medicament bags by a single drive motor could be achieved.

### BRIEF DESCRIPTION OF DRAWINGS

The solution will now be described in more detail by means of exemplifying embodiments and with reference to the accompanying drawings, in which:

FIG. 1 is a schematic illustration of a drug delivery control arrangement, according to possible embodiments.

FIGS. 2A-C are schematic illustrations of transfer mechanisms, according to possible embodiments.

FIGS. 3A-B are schematic illustrations of details, according to possible embodiments.

FIG. 4 is a schematic illustration according to possible embodiments.

FIG. 5 is a schematic illustration of a drug delivery control arrangement, according to possible embodiments.

FIG. 6 is a schematic illustration of a drug delivery control arrangement, according to possible embodiments.

FIG. 7 is a schematic illustration of a drug delivery control arrangement, according to possible embodiments.

FIG. 8 is a schematic illustration of an injector, according to possible embodiments.

### DETAILED DESCRIPTION

Medicaments are commonly delivered in containers, from which they are delivered to patients. The drugs could be pumped to the patients from the containers. Soft, resilient medicament bags are a form of medicament container that is convenient, e.g. because it collapses when being squeezed and does not need any inflow of air to compensate for the medicament leaving the medicament bag, which can avoid contamination of the medicaments. To achieve an appropriate and controlled administration of medicaments, medicament pumps could be arranged to output the medicaments therefrom.

For various medical treatments e.g. of diseases and therapy, the patients require medicament in larger amounts, or the medicament bags comprise different medicaments to be administered in a specific combination and/or order. This disclosure focuses on arrangements for achieving a convenient and effective delivery of medicaments out from a plurality of medicament bags. The actual administration to the patients is a logical following action but will not be further disclosed herein.

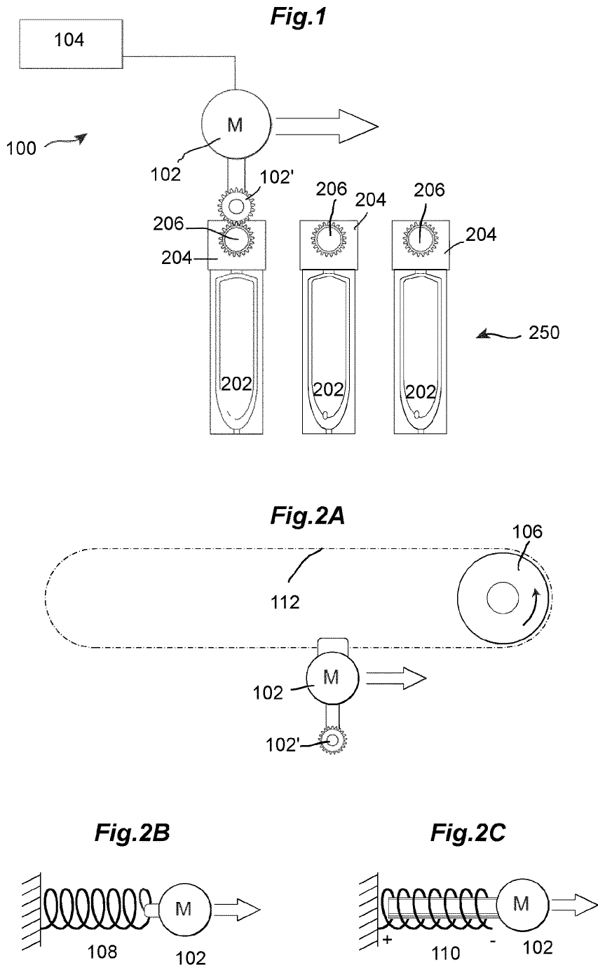

With reference to FIG. 1, which is a schematic illustration, a drug delivery control arrangement will now be described in accordance with an exemplifying embodiment.

The drug delivery control arrangement 100 comprises a drive motor 102 for pumping medicaments out of a plurality of medicament bags 202. In FIG. 1, there are three modules 250 illustrated, where each of the modules 250 comprises a medicament bag 202 and a medicament pump 204. In the figure, the pump 204 is illustrated as a housing with a gear 206. However, the inventive concept is not limited to any specific number of such modules 250, and the drug delivery control arrangement 100 can be configured for delivery of medicaments by medicament pumps 204 from an appropriate number of medicament bags 202.

A control unit 104 of the drug delivery control arrangement 100 is arranged to engage a drive motor 102 with one medicament pump 204 at a time, to deliver medicaments from the corresponding medicament bag 202. When changing medicament pumps, the drive motor 102 disengages from the current delivering medicament pump 204, to engage with another medicament pump 204. The newly engaged medicament pump 204 will then be enabled to deliver medicaments from its corresponding medicament bag 202. Moreover, as illustrated in FIG. 1 and other figures, for illustrative purposes and better understanding, the drive motor 102 is illustrated as having its own driving gear 102′ at a distance from the drive motor's 102 body. However, that is a non-limiting feature, and the skilled designer is free to arrange the drive motor's 102 driving gear 102′ where it is suitable to engage with the medicament pumps 204. It is also to be noted that even if the drive motor 102 is described as having a gear 102′ for engaging with appropriate gears 206 of medicament pumps 204, these gears are a non-limiting example of mechanisms for driving the respective medicament pumps 204. The skilled designer is capable of selecting alternative mechanisms for conveying the driving movements from the drive motor 102. For instance, they may select worm gears or other similar arrangements within the disclosed concept.

By providing an injector with the drug delivery control arrangement 100, the injector is enabled to deliver medicaments from a plurality of medicament bags 202 with only one drive motor 102. As the drive motor 102 is may be noisy and power consuming, it can be beneficial to reduce the number of drive motors. By reducing the number of drive motors, the injector could also be made smaller.

With reference to the FIGS. 2A-C, which are schematic illustrations, some examples of transfer mechanisms for transferring a drive motor between desired positions will now be described in accordance with exemplifying embodiments.

FIG. 2A illustrates an embodiment, where the drive motor 102 is transferred by a control motor 106 and a belt 112. The combination of control motor 106 and belt 112 is a transfer mechanism. When the control motor 106 rotates, the drive motor 102 that is fixed to the belt 112 is moved to change medicament pumps 204 to drive, by disengaging from one medicament pump's gear 206 and instead engaging with another medicament pump's gear 206. The gears 206 are configured to convey the drive motor's 204 power to the engaged one of the medicament pumps 204, to pump medicaments out of corresponding medical bag (not shown). In FIG. 2A, the drive motor 102 moves to the right when the control motor 106 rotates counterclockwise. However, the drive motor 102 may alternatively instead be moved to the left by rotating the control motor 106 clockwise. Thereby, the control motor 106 is capable to drive a plurality of medicament pumps 204, one at a time, and in an appropriate order, to pump medicaments out of corresponding medicament bags 202. Thereby, a flexible sequence of delivering medicaments is achieved.

It is to be noted that the design of the transfer mechanism is described in a non-limiting manner and that also alternative implementations could be applied within the inventive concept. For instance, instead of the belt 112, a wire, or a suitable toothed rack could be applied for conveying the rotating movement of the control motor 106 to a linear transferring movement of the drive motor 102.

FIG. 2B illustrates another embodiment, where the drive motor 102 instead is forced by a spring 108 from one end to another, i.e. a transfer mechanism in form of the spring 108. The spring 108 may be pre-loaded, e.g. by being pressed together before use, and then activated by medical staff or a patient. Typically, the activating person initiates delivery from a first medicament bag, then a second medicament bag, then a third medicament bag, etc., by releasing appropriate flexible stops (not shown), one at a time, such that the drive motor 102 engages with the appropriate medicament pump 204. Such flexible stops may be implemented as mechanical arms that the person releases. In this embodiment, the control unit 104 is mechanically operated and implemented without need for further electric motors than the drive motor 102 itself. Delivery from a first medicament bag, followed by a second medicament bag, followed by the first medicament bag again, would also be an option, as a given bag does not have to be fully emptied; this is also the case for other embodiments described herein.

FIG. 2C illustrates yet another embodiment, where the drive motor 102 instead is forced by a solenoid 110 as a transfer mechanism. The solenoid 110 is controlled by the control unit 104. The control unit 104 generates a voltage over the solenoid 110 that forces the drive motor 102 to engage with the medicament pump 204 of the appropriate medicament bag 202. By generating an appropriate voltage over the solenoid 110, the control unit 104 is enabled to engage the drive motor with the desired medicament pump 204 with precision. The order that the medicament pumps 204 are driven in is flexible, and the control unit 104 may force the drive motor 102 to return to an already left medicament pump 204 when desired.

The transferring mechanisms described above may typically comprise further components to achieve proper functionality. For instance, the drive motor 102 may be transferred along a track between the positions where it will serve the different medicament pumps 204.

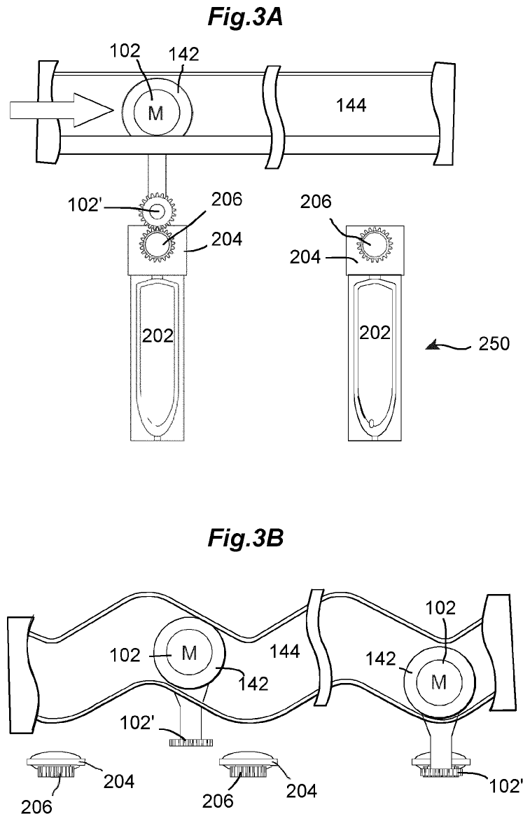

With reference to FIGS. 3A-B, which are schematic illustrations, details of such a track will now be described in accordance with some exemplifying embodiments.

In these figures, the control unit is not shown, as it already has been described above. However, also in this embodiment, the transfer of the drive motor 102 is controlled by a corresponding control unit.

In FIG. 3A, one can see how the drive motor 102 is transferred along a guiding rail or track 144. This guiding rail or track 144 is arranged to achieve a precise movement of the drive motor 102, such that its own gear 102′ engages with the appropriate medicament pump's 204 gear 206 with precision. When the drive motor's gear 102′ disengages with the gear 206 to the left in the figure, the drive motor 102 is guided by the track 144 to the right in the figure, where its gear 102′ engages with the gear 206 of another medicament pump 204. Thereby, the drive motor 102 could stop driving the left medicament pump 204 and instead start to drive the right medicament pump 204 in a reliable sequence, i.e. stop delivering medicaments from the left medicament bag 202 but start to deliver from the right medicament bag.

In FIG. 3B, an alternative design of the track 144 is implemented. The track 144 is non-linear to facilitate proper disengagement/engagement with the gears 206 of the appropriate medicament pump. When the drive motor 102 is forced to the right in FIG. 3B, it follows a non-linear (for example zigzag-shaped or wave-formed) track 144 and moves also up and down in a sequence along the transfer from left to right. This functionality achieves a type of clutch mechanism and may decrease mechanical wear of the gears 102′, 206.

When being guided by a rail and a track 144, the drive motor 102 may have a guide block 142 arranged for various reasons. For instance, the guide block 142 may improve slide characteristics; it may protect the drive motor 102 from mechanical wear; it may adapt the drive motor's 102 dimensions to the track 144, etc. The guide block 142 is implemented as a frame that encompasses the drive motor's 102 body, at least partially, to adapt the size and shape to the track 144. Such a guide block 142 may also be implemented in conjunction with other embodiments of this disclosure, e.g. in the embodiments described with reference to FIG. 3A.

In the above-described exemplifying embodiments, the medicament bags 202 have been arranged in modules 250 and corresponding medicament pumps 204 have been pumping medicaments out from the medicament bags 202. However, the inventive concept is not limited to delivering medicaments from medicament bags arranged in separate modules. Below, an exemplifying embodiment where the drug delivery control arrangement 100 delivers medicaments from a plurality of medicament bags arranged together is provided.

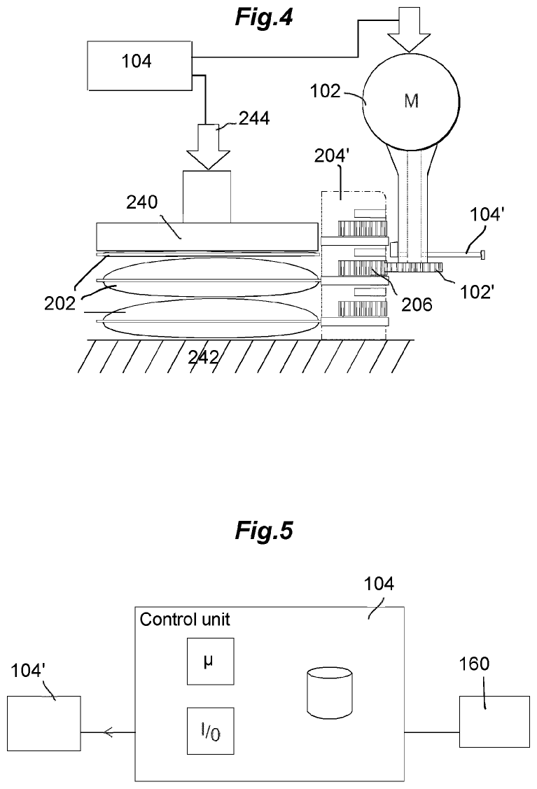

FIG. 4 is a schematic illustration, where three medicament bags 202 are stacked between a first plate 240 and a second plate 242. There are three medicament pumps arranged together in a pump module 204′. In the figure a situation is shown where a first medicament bag 202 has been emptied and the drive motor 102 has been transferred to start driving a second medicament pump of the pump module 204′. The gear 102′ of the drive motor's 102 has engaged with the gear 206 of the second medicament pump.

An actuator 244 is arranged to press the first plate 240 and the second plate 242 together. The medication bag 202 is than squeezed between the plates 240, 242 by the actuator 244. Thus, the first plate 240 together with the second plate 242 and the actuator constitutes a squeeze mechanism. The position of the first plate 240 is determined by a control unit 104 to recognize the dimension change of the current delivering medicament bag 202. In this embodiment, the drug delivery control arrangement is configured to initiate transfer of the drive motor 102 when a current delivering medicament bag 202 is empty, e.g. when the thickness of the current delivering medicament bag 202 is determined to be close to zero. Although the actuator is shown as attached to the control unit 104, the actuator could alternatively be a passive feature such as a spring or elastic, or could be replaced by a gravity-based system in which the plate 240 moves closer to the plate 242 as the drug(s) is/are delivered due to gravity.

The transfer movement of the drive motor 102 is controlled by the control unit 104 and is indicated by an unfilled arrow. To further achieve a more precise movement of the drive motor 102 the control unit 104 may have a transfer actuator 104′ for disengaging and engaging the drive motor 102 with the appropriate medicament pumps 204.

It is to be noted that the parameters used for determining statuses of the drug delivery control arrangement 100 are not limited to dimension change of medicament bags. The skilled designer may choose to measure appropriate parameters related to the currently delivering medicament bag, or the drug delivery system in general, by arranging appropriate sensors for the medicament bags, for the medicament pumps 204, for the drive motor 102, or where appropriate in the drug delivery control arrangement. In particular, parameters related to remaining drug volume and/or remaining drug quantity may be of interest. Some non-limiting examples of relevant parameters for status determination are: remaining drug volume in the currently delivering medicament bag; remaining to-be-injected drug volume in the currently delivering medicament bag; remaining drug quantity in the currently delivering medicament bag; remaining to-be-injected drug quantity in the currently delivering medicament bag; remaining injection time (e.g. based on flow rate or change in bag dimension). Flow rate/speed of injection (and also bag parameters and torque) could also be statuses after being directly measured.

It is to be noted that each of the above exemplified measurements does not need a respective dedicated sensor, instead the control unit 104 may apply some sensors for more than one measurement. Thereby, the number of sensors may be reduced, which may result in a less complex design of the drug delivery control arrangement. For example, flow rate may be determined from change in bag dimensions and time elapsed, or bag dimension may be determined from flow rate and time elapsed. The control unit 104 may further infer parameters indirectly from appropriate sensors, to reduce the number of sensors required, or as a cross-check to provide two independent indications for a particular status.

With reference to FIG. 5, which is a schematic illustration, a drug delivery control arrangement will now be described in accordance with an exemplifying embodiment.

The drug delivery control arrangement comprises a control unit 104 and a drive motor. The drive motor is configured to disengage and engage a plurality of medicament pumps to deliver medicaments out of corresponding medicament bags. The drive motor, the medicament pumps, and the medicament bags correspond to the ones described above in conjunction with other related embodiments and will not be further discussed for this embodiment. Instead, the focus is on describing the implementation of sensors and the control unit 104. The drug delivery control arrangement 100 has one or more sensors 160 arranged at suitable positions of an injector, the one or more sensors being configured to measure parameters related to the currently delivering drug bag. Some examples of appropriate parameters to measure have been defined above.

The control unit 104 comprises a plurality of components for implementing various functionalities and to operate properly. In this embodiment, the control unit comprises components: for receiving signals from the sensors 160; for processing the received signals to determine a status of the delivery of medicaments; and for controlling an injector based on the determined status.

These functionalities are implemented as a communication circuit and a processor, marked as “I/O” (Input/Output) and “μ”, respectively. The control unit 104 may further comprise a memory.

The memory enables the control unit 104 to store information, e.g. regarding statuses of medicament bags, the patient, and earlier injections. The stored information may be used as further input by the processor for when controlling the injector.

The above-described arrangements may be comprised in medical injectors to enable administration of medicaments from a plurality of medicament bags. In accordance with one exemplifying embodiment, a medical injector for administering one or more medication drugs to a patient comprises an injector needle (or another medicament delivery member, such as a catheter or a jet injector), a drug delivery control arrangement according to any of the above defined embodiments, and a hose connecting the injector needle (medicament delivery member) to the drug delivery control arrangement. The hose is configured for delivering medication drugs from two or more medicament bags. Injection may be subcutaneous, intramuscular or intravenous.

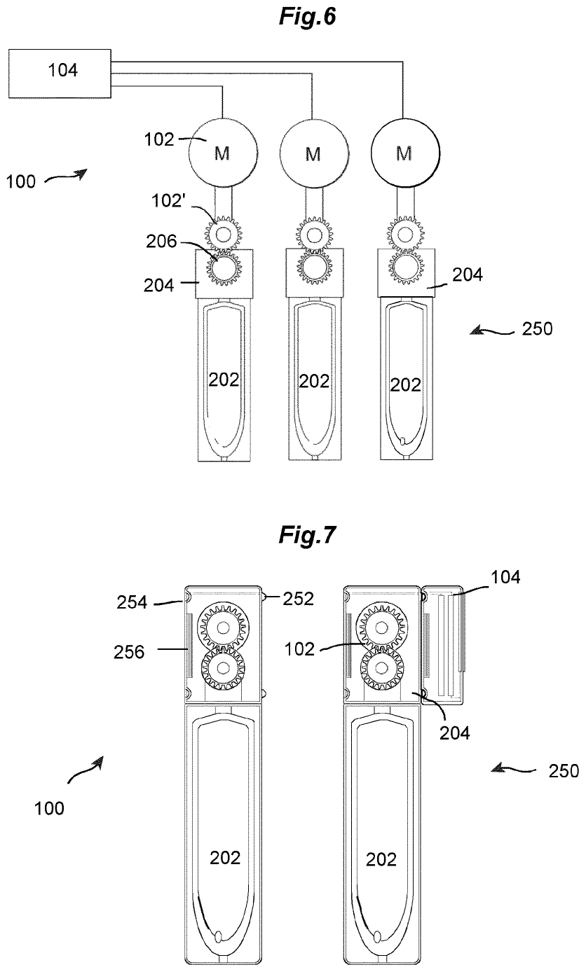

With reference to FIG. 6, which is a schematic illustration, an alternative drug delivery control arrangement will now be described in accordance with one exemplifying embodiment. The drug delivery control arrangement 100 is related to the embodiment illustrated in FIG. 1. However, instead of having one single drive motor 102 that is transferred between the medicament pumps 204, this alternative drug delivery control arrangement 100 comprises individual drive motors 102 for serving the respective medicament pumps 204. The control unit 104 controls which medicament pump 204 is operating and active by sending control signals to the appropriate drive motors 102. The module 250 comprises the medicament pump 204 and has a gear 206 that is engaged with the corresponding gear 102′ of the drive motor 102.

Electrical lines between the control unit 104 and the respective drive motors 102 are arranged. However, the control signals may be alternatively conveyed within the disclosed concept. For instance, the control signals may be transmitted wirelessly by radio or induction.

With reference to FIG. 7, which is a schematic illustration, a drug delivery control arrangement will now be described in accordance with one exemplifying embodiment.

In the figure, the drug delivery control arrangement 100 is illustrated as two modules 250 that are stacked together with a control unit 104. The modules 250 have been described above in another embodiment. Each module 250 has a medicament bag 202 and medicament pump 204. One can see the drive motors 102 that here are engaged with gears 206 of the respective medicament pumps 204. In this embodiment, each module 250 comprises one respective drive motor 102, and the control unit 104 controls which drive motor 102 is active, i.e. which module 250 is currently delivering medicaments from its medicament bag 202.

The modules 250 are provided with guiding means in form of knobs and recesses 252, 254 that fit into each other, and fixating means 256 for keeping the modules together with precision. In this embodiment some of the knobs and recesses 252, 254 comprise electrical connectors to convey control signals from the control unit 104 to the appropriate medicament pumps 204. Electrical wires between the connectors are illustrated as dashed lines. The fixating means 256 are implemented as magnets. However, the guiding means 252, 254 and fixating means 256 could be alternatively implemented within the proposed concept. For instance, the guiding means 252, 254 may be designed without electrical connectors, and the control unit 104 may instead convey the control signals wirelessly. The fixating means 256 may also be alternatively implemented, e.g. as Velcro, tape, or sticky areas.

It is to be noted that the disclosed concept is not limited to having one dedicated drive motor 102 for each module 250. Alternatively, the skilled person may implement the drug delivery control arrangement 100 with stackable modules 250, and only one drive motor 102 that is transferred between the modules 250 to serve the respective medicament pumps 204. Thus, an operator is enabled to implement the drug delivery control arrangement 100, either with stackable modules 250 comprising respective drive motors 102, or by stackable modules 250 driven by one drive motor 102 that is transferred between the modules 250.

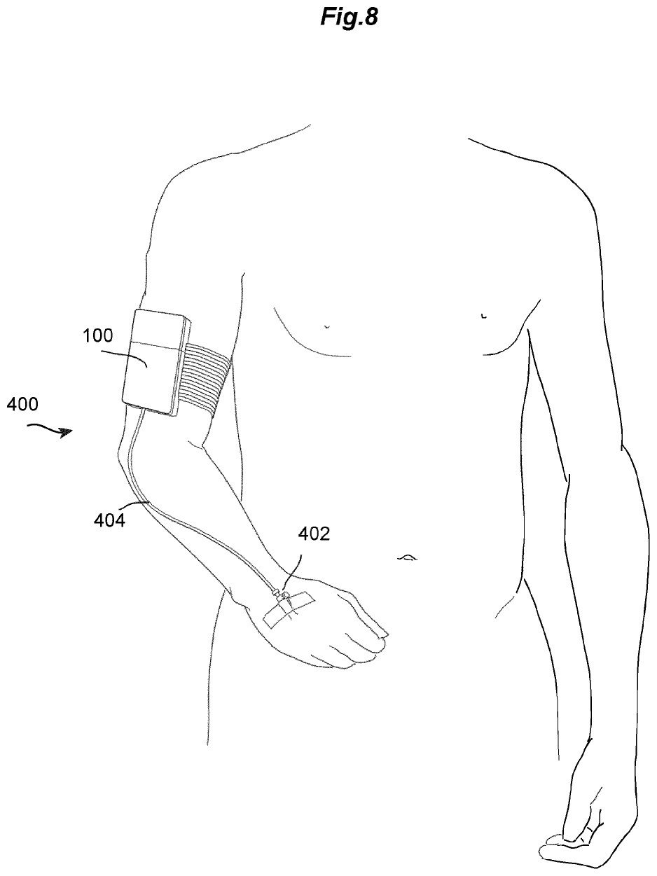

With reference to FIG. 8, which is a schematic overview, an injector for use by a patient will now be described in accordance with an exemplifying embodiment.

The injector 400 comprises a drug delivery control arrangement 100 as defined above in conjunction with other embodiments, a needle 402 (another medicament delivery member, such as a catheter or a jet injector, could instead be provided), and a hose 404. The hose 404 connects the drug delivery control arrangement 100 with the needle 402 and is configured to pump medicaments from medicament bags 202 of the drug delivery control arrangement into the patient to be treated.

In this embodiment, the injector 400 is worn by the patient. As the injector 400 is designed as an on-body device, the patient is not bound to any specific place. Instead the patient could receive the medicaments while doing his/her daily routines at home, and may then be able to live a more natural life. The delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3(LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137(CDw137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6(GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-la, interferon beta-1b, peginterferon beta-la, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC 500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Reference throughout the specification to “one embodiment” or “an embodiment” is used to mean that a particular feature, structure or characteristic described in connection with an embodiment is included in at least one embodiment.

Thus, the appearance of the expressions “in one embodiment” or “in an embodiment” in various places throughout the specification are not necessarily referring to the same embodiment. Further, the particular features, structures or characteristics may be combined in any suitable manner in one or several embodiments. Although the present invention has been described above with reference to specific embodiments, it is not intended to be limited to the specific form set forth herein. Rather, the invention is limited only by the accompanying claims and other embodiments than the specific above are equally possible within the scope of the appended claims. Moreover, it should be appreciated that the terms “comprise/comprises” or “include/includes”, as used herein, do not exclude the presence of other elements or steps.

Furthermore, although individual features may be included in different claims, these may possibly advantageously be combined, and the inclusion of different claims does not imply that a combination of features is not feasible and/or advantageous. In addition, singular references do not exclude a plurality. Finally, reference signs in the claims are provided merely as a clarifying example and should not be construed as limiting the scope of the claims in any way.

The scope is generally defined by the following independent claims. Exemplifying embodiments are defined by the dependent claims.

1-11. (canceled)

12. A drug delivery control arrangement configured to enable medicament delivery from at least two medicament bags, where each one of the medicament bags has a corresponding gear associated for facilitating delivery of the medicament from that medicament bag, the drug delivery control arrangement comprising:
a drive motor configured to be engaged with the gears, one at a time, for delivering the medicament out of the corresponding medicament bags; and
a control unit configured to control from which one of the medicament bags the medicament is delivered by engaging the drive motor with the gear corresponding to that medicament bag.

13. The drug delivery control arrangement according to claim 12, wherein the control unit comprises a transfer mechanism configured to move the drive motor between at least two positions, wherein for each of the positions, the drive motor is engaged with different ones of the gears.

14. The drug delivery control arrangement according to claim 13, wherein the transfer mechanism comprises at least one of: a control motor, a spring, and a solenoid.

15. The drug delivery control arrangement according to claim 13, wherein the transfer mechanism comprises at least one of: a belt, a wire, a toothed rack, and a combination of stops and flexible arms.

16. The drug delivery control arrangement according to claim 13, wherein the transfer mechanism comprises: a track and a guide block, the guide block being fixed to the drive motor, wherein the track and the guide block are configured to guide the drive motor between the at least two positions.

17. The drug delivery control arrangement according to claim 16, wherein the track is non-linear, such that when the guide block is moved along the track, the drive motor is forced by the track to: (i) disengage from one of the gears, and (ii) engage with another one of the gears.

18. The drug delivery control arrangement according to claim 13, further comprising a sensor configured to measure a parameter related to the currently delivering medicament bag when in use, wherein the control unit is configured to initiate movement of the drive motor between the at least two positions based on the measured parameter, the measured parameter being one of: a flow rate out of the currently delivering medicament bag, a torque of the drive motor, and a dimension of the currently delivering medicament bag.

19. The drug delivery control arrangement according to claim 18, further comprising a squeeze mechanism configured to squeeze the at least two medicament bags (202), the squeeze mechanism comprising:
a first plate;
a second plate; and
and an actuator configured to press the first plate and the second plate towards each other, when the medicament bags are stacked between the first plate and the second plate,
wherein the parameter is a distance between the first plate and the second plate.

20. The drug delivery control arrangement according to claim 18, wherein the control unit determines a status of the currently delivering one of the medicament bags based on the measured parameter, and initiates the movement of the drive motor based on the determined status, wherein the status is one of:
remaining drug volume in the currently delivering medicament bag;
remaining drug quantity in the currently delivering medicament bag; and
remaining injection time.

21. An injector for administering at least one medical drug to a patient, comprising:
an injector needle;
a drug delivery control arrangement according claim 12, and
a hose connecting the injector needle to the drug delivery control arrangement, the hose being configured to deliver medicaments from two or more medicament bags.

22. The injector of claim 21, wherein the injector is an on-body device.
